(19) United States
(12) Patent Application Publication
(10) Pub. No.: US 2020/0181599 A1
(43) Pub. Date: Jun. 11, 2020

(54) Immobilized enzyme Pickering emulsion reaction system and application thereof

(71) Applicant: Oil Crops Research Institute,Chinese Academy of Agricultural Sciences, Wuhan (CN)

(72) Inventors: Mingming Zheng, Wuhan (CN); Fenghong Huang, Wuhan (CN); Xia Xiang, Wuhan (CN); Zhe Dong, Wuhan (CN); Chuyun Wan, Wuhan (CN)

(73) Assignee: Oil Crops Research Institute,Chinese Academy of Agricultural Sciences

(21) Appl. No.: 16/749,977

(22) Filed: Jan. 22, 2020

(30) Foreign Application Priority Data
Feb. 1, 2019 (CN) 2019101053333

(51) Int. Cl. C12N 11/14; C12N 9/60; C12N 9/94; C12N 9/20

(52) U.S. Cl. CPC C12N 11/14; C12N 9/60; B82Y 40/00; C12N 9/20; C12N 9/94

(57) ABSTRACT

An immobilized enzyme Pickering emulsion reaction system and application thereof are provided, comprising immobilized enzymes with a mesoporous nanomaterial carrier, an oil phase and an aqueous phase for forming an emulsion, wherein the emulsion has a particle diameter of 10-80 μm, which uses a reaction raw material as the oil phase, uses a butler solution as the aqueous phase, and uses the immobilized enzymes with the mesoporous nanomaterial carrier as both the catalyst and the emulsifier. Compared with conventional emulsions with additional organic reagents or emulsifiers, catalytic activity and stability the Pickering emulsion enzymatic reaction system of the present invention have been significantly improved. Products are easy to separate and purify, easy to reuse, and easy to scale up. The present invention has wider application scope, which is more conducive to environmental protection.

### CROSS REFERENCE OF RELATED APPLICATION

The present invention claims priority under 35 U.S.C. 119(a-d) to CN 201910105333.3, filed Feb. 1, 2019.

### BACKGROUND OF THE PRESENT INVENTION

### Field of Invention

The present invention relates to a technical field of biocatalysis, and more particularly to construction and application of an immobilized enzyme Pickering to emulsion reaction system.

### Description of Related Arts

Enzymes are green and efficient biocatalyst. However, in some high viscosity or heterogeneous reaction systems, free enzyme exhibited poor stability and activity, and is difficult to recycle and reuse, which greatly limits its application in industry. Some researchers have improved the viscosity of the reaction system by adding organic reagents, but there are problems of product separation and residual organic solvents. Pickering emulsion refers to an emulsion formed by using solid particle as an emulsifier, which not only reduces reaction energy and speeds up reaction process, but also facilitates the separation and recovery of emulsifiers and products. Conventionally, immobilized enzymes and Pickering emulsions have been widely used in food, medicine, energy, environment and other fields.

Conventionally, a large number of Pickering emulsion enzymatic reaction systems use enzyme solution or diluted enzyme solution as an aqueous phase, and use additional organic solvent as an oil phase. Different types of materials have been tested to prepare Pickering emulsions, but the additional organic solvent brings tedious separation and purification steps, and is easy to produce residues, which is not suitable for preparation of food-grade products. Chinese patent CN 107955808 A disclosed a method for preparing Pickering emulsion based on double-sided particle stabilization and its application of immobilized enzyme, which uses diluted free enzyme solution and buffer solution as a water phase, resulting in lowered enzyme concentration, smaller contact area between enzyme and substrate and poorer catalytic activity. Chinese patent CN 107973919 A disclosed a method for preparing a dopamine-stabilized Pickering emulsion and its application of immobilized enzyme, which uses the additional dopamine to increase the stability of the emulsion and enzyme, but the cost is high, the operation is complicated, the product is difficult to separate and purify, and the application range is limited.

### SUMMARY OF THE PRESENT INVENTION

To overcome defects of the prior art described above, an object of the present invention is to provide an immobilized enzyme Pickering emulsion reaction system, wherein mesoporous nanomaterial immobilized enzymes act as an emulsifier and a catalyst at the same time, thereby increasing a contact area between enzymes and substrate, and lowering reaction activation energy. Meanwhile, the emulsion directly uses a reaction raw material as an oil phase. Compared with conventional emulsions with additional organic reagents or emulsifiers, catalytic activity and stability are significantly improved. Products are easy to separate and purify, easy to reuse, and easy to scale up. The present invention has wider application scope, which is more conducive to environmental protection.

Accordingly, in order to accomplish the above object, the present invention provides an immobilized enzyme Pickering emulsion reaction system, comprising: immobilized enzymes, an oil phase for forming an emulsion, and an aqueous phase for forming the emulsion, wherein enzymes are immobilized in a mesoporous nanomaterial to form the immobilized enzymes, and the immobilized enzymes are used as both catalysts and emulsifiers; a reaction raw material is used as the oil phase, and a buffer solution is used as the aqueous phase.

Accordingly, the oil phase is the reaction raw material for preparing a target product by using the immobilized enzymes as the catalyst, comprising an esterification reaction raw material, a transesterification reaction raw material, a chiral resolution reaction raw material, and a hydrolysis reaction raw material. For example, the reaction raw material comprises phytosterol and oleic acid for preparing phytosterol oleate, phospholipid and conjugated ethyl linoleate for preparing functionalized phospholipid, 1-phenylethanol and vinyl acetate for chiral resolution of phenylethanol, lauric acid and glycerin for preparing monoglyceride, butyric acid and butanol for preparing butyl butyrate, and olive oil for preparing hydrolyzed olive oil.

Accordingly, the buffer solution has a pH value of 5-8, and a concentration of 0.03 M-0.3 M, which is mainly a phosphate buffer solution or a Tris buffer solution.

Accordingly, the emulsion has a particle size of 10-80 μm; the mesoporous nanomaterial has a particle diameter of 50-500 nm, a specific surface area of 100-700 m²/g, a mesopore size of 8-50 nm, and a loading capacity of 50-600 mg/g for the immobilized enzymes.

Accordingly, the mesoporous nanomaterial is selected from the group consisting of silica particles, carbon particles, organic polymer particles (such as polyester particles), and metal oxide particles (such as TiO₂).

Accordingly, the enzymes comprise porcine pancreatic enzymes, Candida plicata, Candida lipolytica, Candida antarctica, Pseudomonas onion lipase, phospholipase, cellulose, protease or hydrolase.

Accordingly, an emulsifying method comprises using contact probe ultrasound, a handheld homogenizer, a vortex instrument, or a high-pressure homogenizer.

A preparing method of the above immobilized enzyme Pickering emulsion reaction system comprises steps of:

1) preparing a mesoporous nanomaterial according to requirements, and immobilizing enzymes on the mesoporous nanomaterial to form immobilized enzymes;

2) selecting a reaction raw material according to a target product, and then mixing the reaction raw material as an oil phase with a buffer solution as an aqueous phase to form a mixture; dispersing the immobilized enzymes into the mixture as both a catalyst and an emulsifier by ultrasound, so as to obtain a mixed liquid; and

3) using contact probe ultrasound, a handheld homogenizer, a vortex instrument, or a high-pressure homogenizer for emulsifying the mixed liquid obtained in the step 2) to form the immobilized enzyme Pickering emulsion reaction system with a particle size of 10-80 μm.

Accordingly, the immobilized enzyme Pickering emulsion reaction system obtained in the step 3) is reacted at a room temperature or under heating to obtain the target product, wherein after the reaction, the immobilized enzymes in the reaction system can be recovered and reused.

Accordingly, based on the needs of the emulsion, the mesoporous nanomaterial in the step 1) is hydrophilically or hydrophobically modified. For example, nitric acid or sulfuric acid can be used to hydrophilically modified the mesoporous nanomaterial, or a silane coupling agent is used to hydrophobically modify the mesoporous nanomaterial, thereby forming oil-in-water or water-in-oil emulsions by surface hydrophilicity.

Accordingly, in the step 2), an ultrasonic dispersion time is 30-60 s, and an ultrasonic power is 60-120 W.

Accordingly, in the step 2), a mass ratio of the immobilized enzyme, the reaction raw material, and the buffer solution is (0.025-0.01) g:1 g:(0.1-0.5) g;

Accordingly, for emulsifying in the step 3), an ultrasound power of the contact probe ultrasound is preferably (6-50) W/mL and ultrasound intervals are preferably 3 s/9 s-9 s/3 s; a revolving speed of the handheld homogenizer is preferably (10000-30,000) rmp, to and a homogenization time is preferably (2-10) min; an oscillation speed of the vortex instrument is preferably (2500-5000) rpm, and an oscillation time is preferably (5-11.0) min; a pressure of the high-pressure homogenizer is preferably (2000-10000) psi, and a cycle number is preferably 2-4 times.

Compared with the prior art, beneficial effects of the present invention are as follows.

1. The present invention uses the reaction raw materials such as oils and fatty acids as the oil phase without adding any additional organic solvents. The immobilized enzymes are used as both the emulsifier and the enzyme catalyst to construct the Pickering emulsion efficient enzymatic reaction system, which can improve emulsion stability, and avoid instability and low enzyme concentration when adding the Pickering emulsion into the reaction system. The present invention adopts a simple preparing method and simple immobilization steps, and has many applicable types of enzymes. The present invention can be widely used in reactions such as enzymatic esterification, transesterification, and hydrolysis, providing no solvent pollution. Furthermore, the present invention has high catalytic activity, high yield, and high reaction efficiency, wherein the product is easy to separate and purify, easy to reuse, and easy to scale up.

2. The present invention firstly proposes that the mesoporous nano-immobilized enzyme serves as both the catalyst and the emulsifier to prepare the immobilized enzyme Pickering emulsion reaction system, thereby significantly increasing a contact area between enzymes and substrate, as well as lowering reaction activation energy. Meanwhile, the mesoporous nano-immobilized enzyme has a large specific surface and a suitable pore size, which is conducive to enzyme adsorption and substrate mass transfer, thereby speeding up the reaction process.

3. The immobilized enzyme Pickering emulsion reaction system of the present invention is different from other Pickering emulsions where enzyme solution and reactants are randomly distributed. The catalyst-enzyme of the reaction system is mainly distributed at an oil-water interface. The reactants are distributed in the emulsion, which can maximize the enzyme activity, shorten the distance between the reactants and the enzymes, and speed up the reaction process.

### BRIEF DESCRIPTION OF THE DRAWINGS

FIG. 1 is a scanning electron microscope image of mesoporous nano-carbon spheres according to an embodiment 1;

FIG. 2 is a scanning electron microscope image of mesoporous nano-silicon spheres according to an embodiment 2;

FIG. 3 is a scanning electron microscope image of mesoporous nano-titanium spheres according to an embodiment 3;

FIG. 4 is an optical microscope image of an emulsion of an immobilized enzyme Pickering emulsion reaction system according to the present invention (related to the embodiment 1).

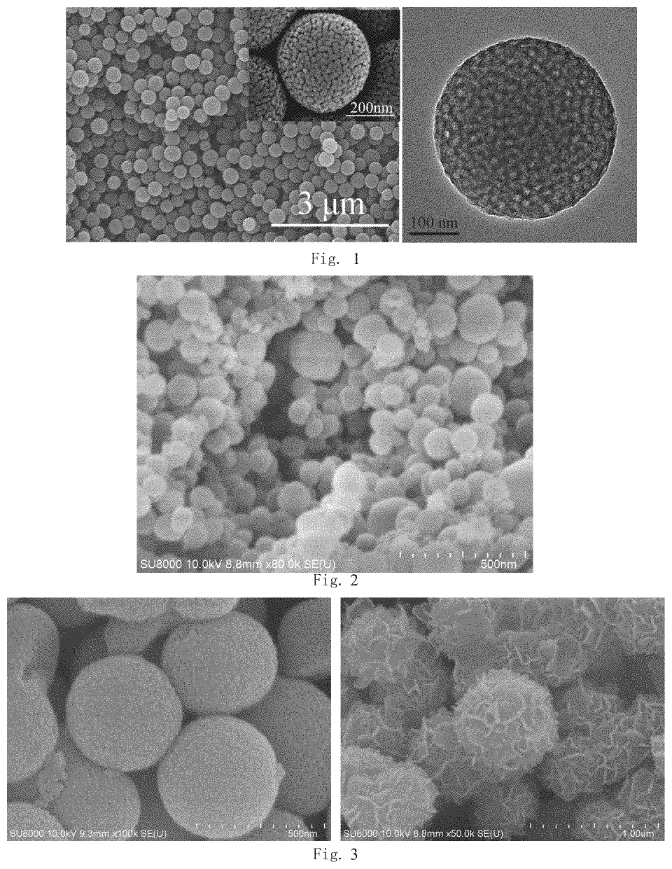

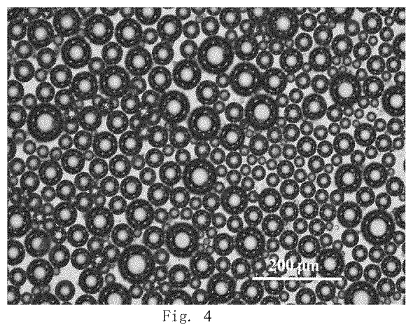

### DETAILED DESCRIPTION OF THE PREFERRED EMBODIMENT

The following embodiments are only used to clarify the content of the present invention in detail and to facilitate a better understanding of the present invention, which are included in the protection scope of the present invention, but not intended to be limiting.

### Embodiment 1

An immobilized enzyme Pickering emulsion reaction system comprises mesoporous nano-carbon sphere-immobilized enzymes, an oil phase and an aqueous phase for forming an emulsion, wherein the emulsion has a particle diameter of 10-80 μm, which uses a reaction raw material of phytosterol and oleic acid (with a mass ratio of 1:4) as the oil phase, uses 0.05M pH=6.5 sodium phosphate buffer solution (PBS) as the aqueous phase, and uses the mesoporous nano-carbon sphere-immobilized enzymes as both a catalyst and an emulsifier. A mass ratio of the nano-carbon sphere immobilized enzymes, the reaction raw material and the phosphate buffer solution is 0.025 g:1g :0.1 g; a mesoporous nano-carbon sphere carrier has a particle diameter of 270-320 nm, a specific surface area of 580-700 m²/g, a mesopore size of 8-14 nm, and an enzyme loading capacity of 100 mg/g.

A preparing method of the immobilized enzyme Pickering emulsion reaction system comprises the following steps of:

1) selecting mesoporous nano-carbon spheres, and observing by electron microscope, wherein the mesoporous nano-carbon spheres have a diameter of 270-320 nm, a specific surface area of 580-700 m²/g, and a mesopore size of 8-1.4 nm; placing 1 g of the mesoporous nano-carbon spheres in 50 mL 49% concentrated sulfuric acid for ultrasound for 1 h, and finally washing with water to neutrality to prepare hydrophilic mesoporous nano-carbon spheres;

2) immobilizing enzymes on the mesoporous nano-carbon spheres: dispersing 0.12 g Candida plicata in 10 mL, 50 mmol, 50 mmol, pH 5.0 sodium phosphate buffer solution to prepare an enzyme solution;

then dispersing the hydrophilic mesoporous nano-carbon spheres into 10 mL of the enzyme solution, and immobilizing the enzymes in a thermostatic oscillator after synchronic ultrasound for 10 min and vacuum for 10 min, wherein a reaction temperature is 4° C., an oscillation speed is 160 rpm, and a reaction time is 0.5 h; after reaction, washing with the buffer solution for 3 times; centrifuging and freeze-drying to obtain the mesoporous nano-carbon sphere-immobilized enzymes, with enzyme loading capacity of 100 mg/g;

3) preparing a phytosterol oleate Pickering emulsion enzymatic reaction system: adding 0.025 g of the mesoporous nano-carbon sphere-immobilized enzymes into a mixed solution of 0.2 g phytosterol, 0.8 g oleic acid reactant and 0.1 g PBS; ultrasonically dispersing for 30 s; homogenizing at 15000 rpm for 2 min with a hand-held homogenizer, to obtain a Pickering emulsion enzymatic reaction system with an emulsion particle size of 10-80 μm; and

4) placing the Pickering emulsion enzyme reaction system obtained in the step 3) in a 55° C. thermostatic water bath and stirring for 4 h at a stirring speed of 300 rpm; after reaction, centrifuging and separating the mesoporous nano-carbon sphere-immobilized enzymes, wherein a sterol esterification rate is 93.7%;

washing the mesoporous nano-carbon sphere-immobilized enzyme separated in step 4) with isooctane for 3 times before recovering, and repeating the above reactions for 10 times, wherein the sterol esterification rate is still higher than 91%.

### Embodiment 2

An immobilized enzyme Pickering emulsion reaction system comprises mesoporous nano-silicon sphere-immobilized enzymes, an oil phase and an aqueous phase for forming an emulsion, wherein the emulsion has a particle diameter of 10-80 μm, which uses a reaction raw material of phospholipid and conjugated ethyl linoleate (with a mass ratio of 1:4) as the oil phase, uses 0.3M pH=6 sodium phosphate buffer solution (PBS) as the aqueous phase, and uses the mesoporous nano-silicon sphere-immobilized enzymes as both a catalyst and an emulsifier. A mass ratio of the nano-silicon sphere immobilized enzymes, the reaction raw material and the phosphate buffer solution is 0.05 g:1 g:0.2 g; a mesoporous nano-silicon sphere carrier has a particle diameter of 200-350 nm, a specific surface area of 180-300 m²/g, a mesopore size of 8-14 nm, and an enzyme loading capacity of 300 mg/g.

A preparing method of the immobilized enzyme Pickering emulsion reaction system comprises the following steps of:

1) preparing a mesoporous silicon nanomaterial, selecting mesoporous nano-silicon spheres, and observing by electron microscope, wherein the mesoporous nano-silicon spheres have a diameter of 200-350 nm, a specific surface area of 180-300 m²/g, and a mesopore size of 8-14 nm;

placing 1 g of the mesoporous nano-silicon spheres in 50 mL toluene for stirring and dispersing; adding 0.5 g octyltrimethoxysilane, and then placing in a reaction kettle for reacting at 100° C. for 24 h, and finally centrifuging to obtain solid powder, so as to prepare hydrophobic mesoporous silicon spheres;

2) immobilizing enzymes on the hydrophobic mesoporous silicon spheres: dispersing 0.4 g phospholipase in 10 mL, 50 mmol, pH 6.5 sodium phosphate buffer solution to prepare an enzyme solution;

then dispersing the hydrophobic mesoporous silicon spheres into 10 mL of the enzyme solution, and immobilizing the enzymes in a thermostatic oscillator after synchronic ultrasound for 10 min and vacuum for 10 min, wherein a reaction temperature is 4° C., an oscillation speed is 160 rpm, and a reaction time is 0.5 h; after reaction, washing with the buffer solution for 3 times; centrifuging and freeze-drying to obtain the mesoporous nano-silicon sphere-immobilized enzymes, with enzyme loading capacity of 300 mg/g;

3) preparing and using a functionalized phospholipid Pickering emulsion enzymatic reaction system: adding 0.05 g of the mesoporous nano-silicon sphere-immobilized enzymes into a mixed solution of 0.2 g phospholipid, 0.8 g conjugated ethyl linoleate reactant and 0.2 g PBS (0.3M pH 6); emulsifying with a contact probe ultrasound with an ultrasound power of 25 W/mL and ultrasound intervals of 6 s/9 s, to obtain a Pickering emulsion enzymatic reaction system with an emulsion particle size of 10-80 μm; and

4) placing the Pickering emulsion enzyme reaction system obtained in the step 3) in a 55° C. thermostatic water bath and reacting for 4 h at a stirring speed of 300 rpm; after reaction, centrifuging and removing the immobilized enzymes, wherein a sterol esterification rate is 95.7%;

washing the mesoporous nano-silicon sphere-immobilized enzyme separated in step 4) with n-heptane for 2 times before recovering, and repeating the above reactions for 10 times, wherein the sterol esterification rate is still higher than 90%.

### Embodiment 3

An immobilized enzyme Pickering emulsion reaction system comprises mesoporous nano-titanium sphere-immobilized enzymes, an oil phase and an aqueous phase for forming an emulsion, wherein the emulsion has a particle diameter of 10-80 μm, which uses a reaction raw material of vinyl acetate and 1-phenylethanol (with a mass ratio of 4:1) as the oil phase, uses 0.05M pH=8 sodium phosphate buffer solution (PBS) as the aqueous phase, and uses the mesoporous nano-titanium sphere-immobilized enzymes as both a catalyst and an emulsifier. A mass ratio of the nano-titanium sphere immobilized enzymes, the reaction raw material and the phosphate butler solution is 0.05 g:4.66 g:0.428 g; a mesoporous nano-titanium sphere carrier has a particle diameter of 200-350 nm, a specific surface area of 100-200 m²/g, a mesopore size of 8-14 nm, and an enzyme loading capacity of 200 mg/g.

A preparing method of the immobilized enzyme Pickering emulsion reaction system comprises the following steps of:

1) preparing and selecting mesoporous nano-titanium spheres, and observing by electron microscope, wherein the mesoporous nano-titanium spheres have a diameter of 200-350 nm, a specific surface area of 100-200 m²/g, and a mesopore size of 8-14 nm;

2) immobilizing enzymes on the mesoporous nano-titanium spheres: dispersing 0.4 g Pseudomonas onion lipase in 10 mL, 50 mmol, pH 6.5 phosphate buffer solution to prepare an enzyme solution;

then dispersing the mesoporous titanium spheres into 10 mL of the enzyme solution, and immobilizing the enzymes in a thermostatic oscillator after synchronic ultrasound for 10 min and vacuum for 10 min, wherein a reaction temperature is 4° C., an oscillation speed is 160 rpm, and a reaction time is 0.5 h; after reaction, washing with the buffer solution for 3 times; centrifuging and freeze-drying to obtain the mesoporous nano-titanium sphere-immobilized enzymes, with enzyme loading capacity of 200 mg/g;

3) preparing and using a phenylethanol chiral resolution Pickering emulsion enzymatic reaction system: weighing 1.22 g 1-phenylethanol, and weighing vinyl acetate according to a molar ratio of vinyl acetate to 1-phenylethanol of 4:1; adding 0.05 g of the mesoporous nano-titanium sphere-immobilized enzymes into a mixed solution of 1-phenylethanol, vinyl acetate and 0.428 g PBS (0.05M pH 8); ultrasonically dispersing for 30 s; oscillating at 15000 rpm for 2 min with a vortex instrument, to obtain a Pickering emulsion enzymatic reaction system with an emulsion particle size of 10-80 μm; and

4) placing the Pickering emulsion enzyme reaction system obtained in the step 3) in a 55° C. thermostatic water bath and reacting for 6 h at a stirring speed of 300 rpm; after reaction, centrifuging and removing the immobilized enzymes, wherein a phenylethanol esterification rate is 50%;

washing the mesoporous nano-titanium sphere-immobilized enzyme separated in step 4) with acetone for 2 times before recovering, and repeating the above reactions for 10 times, wherein the phenylethanol esterification rate is still about 50%, and reusability is sufficient.

### Embodiment 4

An immobilized enzyme Pickering emulsion reaction system comprises petal-shaped mesoporous nano-titanium sphere-immobilized enzymes, an oil phase and an to aqueous phase for forming an emulsion, wherein the emulsion has a particle diameter of 10-80 μm, which uses a reaction raw material of lauric acid and glycerin (with a mass ratio of 1:2) as the oil phase, uses potassium phosphate buffer solution (0.1M pH=7.5) as the aqueous phase, and uses the petal-shaped mesoporous nano-titanium sphere-immobilized enzymes as both a catalyst and an emulsifier. A mass ratio of the petal-shaped nano-titanium sphere immobilized enzymes, the reaction raw material and the phosphate buffer solution is 0.05 g:0.6 g:0.428 g; a mesoporous nano-titanium sphere carrier has a particle diameter of 200-350 nm, a specific surface area of 100-300 m²/g, a mesopore size of 8-14 nm, and an enzyme loading capacity of 50 mg/g.

A preparing method of the immobilized enzyme Pickering emulsion reaction system comprises the following steps of:

1) preparing and selecting petal-shaped mesoporous nano-titanium spheres, and observing by electron microscope, wherein the petal-shaped mesoporous nano-titanium spheres have a diameter of 200-350 nm, a specific surface area of 100-300 m²/g, and a mesopore size of 8-14 nm;

2) immobilizing enzymes on the petal-shaped mesoporous nano-titanium spheres: dispersing 0.4 g porcine pancreatic enzyme in 10 mL, 40 mmol, pH 7 phosphate buffer solution to prepare an enzyme solution;

then dispersing the petal-shaped mesoporous titanium spheres into 10 mL of the enzyme solution, and immobilizing the enzymes in a thermostatic oscillator after synchronic ultrasound for 10 min and vacuum for 10 min, wherein a reaction temperature is 4° C., an oscillation speed is 160 rpm, and a reaction time is 0.5 h; after reaction, washing with the buffer solution for 3 times; centrifuging and freeze-drying to obtain the petal-shaped mesoporous nano-titanium sphere-immobilized enzymes, with enzyme loading capacity of 50 mg/g;

3) preparing and using a monoglyceride Pickering emulsion enzymatic reaction system: adding 0.05 g of the petal-shaped mesoporous nano-titanium sphere-immobilized enzymes into 0.2 g lauric acid, 0.4 g glycerin and 0.428 g potassium phosphate buffer solution (0.1M pH=7.5); ultrasonically dispersing for 30 s; homogenizing at 600 psi for 2 times with a high-pressure homogenizer, to obtain a Pickering emulsion enzymatic reaction system with an emulsion particle size of 10-80 μm; and

4) placing the Pickering emulsion enzyme reaction system obtained in the step 3) in a 55° C. thermostatic water bath and reacting for 6 h at a stirring speed of 300 rpm; after reaction, centrifuging and removing the immobilized enzymes, wherein a monoglyceride esterification rate is 95%;

washing the petal-shaped mesoporous nano-titanium sphere-immobilized enzyme separated in step 4) with isooctane for 2 times before recovering, and repeating the above reactions for 10 times, wherein the monoglyceride esterification rate is still about 95%.

### Embodiment 5

An immobilized enzyme Pickering emulsion reaction system comprises double-layer hollow carbon sphere-immobilized enzymes, an oil phase and an aqueous phase for forming an emulsion, wherein the emulsion has a particle diameter of 10-80 μm, which uses a reaction raw material of butyric acid and butanol (with a mass ratio of 0.45:0.55) as the oil phase, uses 0.03M pH=5 Tris buffer solution as the aqueous phase, and uses the double-layer hollow carbon sphere-immobilized enzymes as both a catalyst and an emulsifier. A mass ratio of the nano-carbon sphere immobilized enzymes, the reaction raw material and the phosphate buffer solution is 0.05 g:1 g:0.3 g; the double-layer hollow carbon sphere-immobilized enzymes have a particle diameter of 200-350 nm, a specific surface area of 400-600 m²/g, a mesopore size of 8-14 nm, and an enzyme loading capacity of 400 mg/g.

A preparing method of the immobilized enzyme Pickering emulsion reaction system comprises the following steps of:

1) preparing double-layer hollow carbon spheres, and observing by electron microscope, wherein the double-layer hollow carbon spheres have a diameter of 200-350 nm, a specific surface area of 400-600 m²/g, and a mesopore size of 8-14 nm;

2) immobilizing enzymes on the double-layer hollow carbon spheres: dispersing 0.4 g Pseudomonas onion lipase in 10 mL, 50 mmol, pH 6.5 sodium phosphate buffer solution to prepare an enzyme solution;

then dispersing the hydrophilic double-layer hollow carbon spheres into 10 mL of the enzyme solution, and immobilizing the enzymes in a thermostatic oscillator after synchronic ultrasound for 10 min and vacuum for 10 min, wherein a reaction temperature is 4° C., an oscillation speed is 160 rpm, and a reaction time is 2.5 h; after reaction, washing with the buffer solution for 3 times; centrifuging and freeze-drying to obtain the double-layer hollow carbon sphere-immobilized enzymes, with enzyme loading capacity of 400 mg/g;

3) preparing and using a butyl butyrate Pickering emulsion enzymatic reaction system: adding 0.05 g of the double-layer hollow carbon sphere-immobilized enzymes into a mixed solution of 0.45 g butyric acid, 0.55 g butanol reactant and 0.3 g Tris buffer solution (0.03M pH=5); ultrasonically dispersing for 30 s; homogenizing at 6000 rpm for 3 min with a hand-held homogenizer, to obtain a Pickering emulsion enzymatic reaction system with an emulsion particle size of 10-80 μm; and

4) placing the Pickering emulsion enzyme reaction system obtained in the step 3) in a 37° C. thermostatic water bath and reacting for 6 h at a stirring speed of 300 rpm; after reaction, centrifuging and removing the immobilized enzymes, wherein a butanol esterification rate is 95.7%;

washing the double-layer hollow carbon sphere-immobilized enzyme separated in step 4) with acetone for 2 times before recovering, and repeating the above reactions for 10 times, wherein the butanol esterification rate is still higher than 90%.

### Embodiment 6

An immobilized enzyme Pickering emulsion reaction system comprises double-layer hollow carbon sphere-immobilized enzymes, an oil phase and an aqueous phase for forming an emulsion, wherein the emulsion has a particle diameter of 10-80 ∥m, which uses a reaction raw material of butyric acid and butanol (with a mass ratio of 0.45:0.55) as the oil phase, uses 0.02M pH-7.5 PBS buffer solution as the aqueous phase, and uses the double-layer hollow carbon sphere-immobilized enzymes as both a catalyst and an emulsifier. A mass ratio of the nano-carbon sphere immobilized enzymes, the reaction raw material and the phosphate buffer solution is 0.05 g:2 mL:0.3 g; the double-layer hollow carbon sphere-immobilized enzymes have a particle diameter of 200-350 nm, a specific surface area of 400-600 m²/g, a mesopore size of 8-14 nm, and an enzyme loading capacity of 600 mg/g.

A preparing method of the immobilized enzyme Pickering emulsion reaction system comprises the following steps of:

1) preparing double-layer hollow carbon spheres, and observing by electron microscope, wherein the double-layer hollow carbon spheres have a diameter of 200-350 nm, a specific surface area of 400-600 m²/g, and a mesopore size of 8-14 nm;

2) immobilizing enzymes on the double-layer hollow carbon spheres: dispersing 0.4 g porcine pancreatic enzyme in 10 mL, 50 mmol, pH 6.5 sodium phosphate buffer solution to prepare an enzyme solution;

then dispersing the hydrophilic double-layer hollow carbon spheres into 10 mL of the enzyme solution, and immobilizing the enzymes in a thermostatic oscillator after synchronic ultrasound for 10 min and vacuum for 10 min, wherein a reaction temperature is 4° C., an oscillation speed is 160 rpm, and a reaction time is 2.5 h; after reaction, washing with the buffer solution for 3 times; centrifuging and freeze-drying to obtain the double-layer hollow carbon sphere-immobilized enzymes, with enzyme loading capacity of 600 mg/g;

3) preparing and using a hydrolyzed olive oil Pickering emulsion enzymatic reaction system: adding 0.05 g of the double-layer hollow carbon sphere-immobilized enzymes into a mixed solution of 2 mL and 0.3 g PBS buffer solution (0.02M pH=7.5); ultrasonically dispersing for 30 s; homogenizing at 20000 rpm for 3 min with a hand-held homogenizer, to obtain a Pickering emulsion enzymatic reaction system with an emulsion particle size of 10-80 μm; and

4) placing the Pickering emulsion enzyme reaction system obtained in the step 3) in a 37° C. thermostatic water bath and reacting for 6 h at a stirring speed of 400 rpm; after reaction, centrifuging and removing the immobilized enzymes, wherein an olive oil hydrolysis rate is 90%;

washing the double-layer hollow carbon sphere-immobilized enzyme separated in step 4) with N-hexane for 2 times before recovering, and repeating the above reactions for 10 times, wherein the hydrolysis rate is still higher than 85%.

### Embodiment 7

The embodiment 7 is basically the same as the embodiment 5, except for that the double-layer hollow carbon spheres in the step 1) are replaced by hollow carbon spheres; and Pseudomonas onion lipase in the step 2) is replaced by Candida antarctica.

### Embodiment 8

The embodiment 8 is basically the same as the embodiment 5, except for that the double-layer hollow carbon spheres in the step 1) are replaced by cyclodextrin polymer; and Pseudomonas onion lipase in the step 2) is replaced by porcine pancreatic enzyme.

The above is only preferred embodiments of the present invention. It should be noted that, for those of ordinary skill in the art, without departing from the inventive concept of the present invention, several improvements and modifications can be made. Such improvements and modifications all belong to the protection scope of the present invention.

What is claimed is:

1. An immobilized enzyme Pickering emulsion reaction system, comprising: an oil phase for forming an emulsion, an aqueous phase for forming the emulsion, and immobilized enzymes, wherein enzymes are immobilized in a mesoporous nanomaterial to form the immobilized enzymes, and the immobilized enzymes are used as both a catalyst and an emulsifier; a reaction raw material is used as the oil phase, and a buffer solution is used as the aqueous phase.

2. The immobilized enzyme Pickering emulsion reaction system, as recited in claim 1, wherein the oil phase is the reaction raw material for preparing a target product by using the immobilized enzymes as the catalyst, comprising an esterification reaction raw material, a transesterification reaction raw material, a chiral resolution reaction raw material, and a hydrolysis reaction raw material.

3. The immobilized enzyme Pickering emulsion reaction system, as recited in claim 2, wherein the reaction raw material comprises phytosterol and oleic acid for preparing phytosterol oleate, phospholipid and conjugated ethyl linoleate for preparing functionalized phospholipid, 1-phenylethanol and vinyl acetate for chiral resolution of phenylethanol, lauric acid and glycerin for preparing monoglyceride, butyric acid and butanol for preparing butyl butyrate, and olive oil for preparing hydrolyzed olive oil.

4. The immobilized enzyme Pickering emulsion reaction system, as recited in claim 1, wherein the buffer solution has a pH value of 5-8, and a concentration of 0.03M-0.3M.

5. The immobilized enzyme Pickering emulsion reaction system, as recited in claim 1, wherein the emulsion has a particle size of 10-80 μm.

6. The immobilized enzyme Pickering emulsion reaction system, as recited in claim 1, wherein the mesoporous nanomaterial is selected from the group consisting of silica particles, carbon particles, polymer particles, and metal oxide particles; the mesoporous nanomaterial has a particle diameter of 50-500 nm, specific surface area of 100-700 m²/g, a mesopore size of 8-50 nm, and a loading capacity of 50-600 mg/g for the immobilized enzymes.

7. A preparing method of an immobilized enzyme Pickering emulsion reaction system as recited in claim 1, comprising steps of:
1) preparing a mesoporous nanomaterial according to requirements, and immobilizing enzymes on the mesoporous nanomaterial to form immobilized enzymes;
2) selecting a reaction raw material according to a target product, and then mixing the reaction raw material as an oil phase with a buffer solution as an aqueous phase to form a mixture; dispersing the immobilized enzymes into the mixture as both a catalyst and an emulsifier by ultrasound, so as to obtain a mixed liquid; and
3) emulsifying the mixed liquid obtained in the step 2) to form the immobilized enzyme Pickering emulsion reaction system with a particle size of 10-80 μm.

8. The preparing method, as recited in claim 7, wherein the immobilized enzyme Pickering emulsion reaction system obtained in the step 3) is reacted at a room temperature or under heating to obtain the target products.

9. The preparing method, as recited in claim 7, wherein in the step 2), a mass ratio of the immobilized enzyme, the reaction raw material, and the buffer solution is (0.025-0.01) g:1 g:(0.1-0.5) g; in the step 2), an ultrasonic dispersion time is 30-60 s, and an ultrasonic power is 60-120 W.

10. The preparing method, as recited in claim 7, wherein in the step 3), an emulsifying method comprises using contact probe ultrasound, a handheld homogenizer, a vortex instrument, or a homogenizer.
